(19) United States
(12) Patent Application Publication
(10) Pub. No.: US 2016/0339126 A1
(43) Pub. Date: Nov. 24, 2016

(54) ULTRAVIOLET DISINFECTION SYSTEM FOR ATHLETIC ITEMS

(71) Applicant: George J. Lichtblau, New Canaan, CT (US)

(72) Inventor: George J. Lichtblau, New Canaan, CT (US)

(21) Appl. No.: 14/714,852

(22) Filed: May 18, 2015

(51) Int. Cl. A61L 2/10; A61L 2/24

(52) U.S. Cl. CPC A61L 2/10; A61L 2/24

(57) ABSTRACT

An ultraviolet disinfection system to generate UVC radiation inside athletic clothing, gear and other items to be decontaminated. The system includes a plurality of UVC lamps vertically disposed in a housing and having UVC transmissive lamp enclosures over which items to be decontaminated are placed. The system has a microprocessor based controller and can kill pathogens including Clostridium difficile in 30 seconds or less.

### STATEMENT REGARDING FEDERALLY SPONSORED RESEARCH OR DEVELOPMENT

N/A

### BACKGROUND OF THE INVENTION

Clothing and protective gear worn by athletes such as helmets, gloves, shoes, sneakers and socks provide an environment conducive to the rapid breeding of pathogens since the interior of these items are warm from body heat, moist from body perspiration and dark because external light does not enter the interior of these items.

It is known that UVC radiation is effective in killing or deactivating pathogens in air, water and exposed surfaces. A system for providing UVC radiation to kill pathogens in the air and on radiated surfaces in a room is shown in U.S. Pat. No. 8,791,441 of the same inventor as the present invention.

It would be useful to have an effective and convenient system for decontamination of pathogens in the interior of athletic clothing, gear and other such items.

### BRIEF SUMMARY OF THE INVENTION

The present invention provides an ultraviolet radiation system which generates UVC radiation inside of objects to be decontaminated. UVC radiation having a prime wavelength of 253.7 nm (referred to as 254 nm) is provided by low pressure high output mercury or amalgam UVC lamps. One or more lamps may be employed in embodiments of the novel system. In one embodiment, a plurality of UVC lamps is contained within an enclosure or housing, with each lamp being vertically disposed and surrounded by UVC transparent lamp enclosure such as an open grid or basket structure or UVC transparent tube which may be sized and configured to accommodate specific items such as gloves, shoes, sneakers, helmets and the like. The athletic items are placed over respective lamp enclosures such that each UVC lamp is substantially inside an item to permit UVC radiation to kill pathogens inside the item. The UVC lamps are of U shape having connectors on one end that can be plugged into electrical lamp sockets in the housing and easily plugged in and out for replacement. Preferably each lamp is covered by a protective sleeve to avoid shattering of the lamp glass in the event of breakage. FEP (Teflon) is preferred because it is UVC transmissive with little attenuation and can easily withstand the operating temperature of the UVC lamps. The UVC radiation intensity provided by the lamps is sufficiently strong to decontaminate pathogens including Clostridium difficile in 30 seconds or less. The on time of the lamps is so short that self-heating of the lamps has no effect on performance of the system.

### BRIEF DESCRIPTION OF THE SEVERAL VIEWS OF THE DRAWINGS

The invention will be more fully understood from the following detailed description in conjunction with the drawings in which:

FIG. 1 is a pictorial view of one embodiment of a system in accordance with the invention;

FIG. 2 is a pictorial view of an open grid enclosure used in the invention;

FIGS. 3a-3c are illustrations of typical items for use in the invention;

FIG. 4 is a pictorial view of a quartz tube enclosure used in the invention;

FIG. 5 is a diagrammatic view of a control panel of the embodiment of FIG. 1; and

FIG. 6 is a block diagram of a system in accordance with the invention.

### DETAILED DESCRIPTION OF THE INVENTION

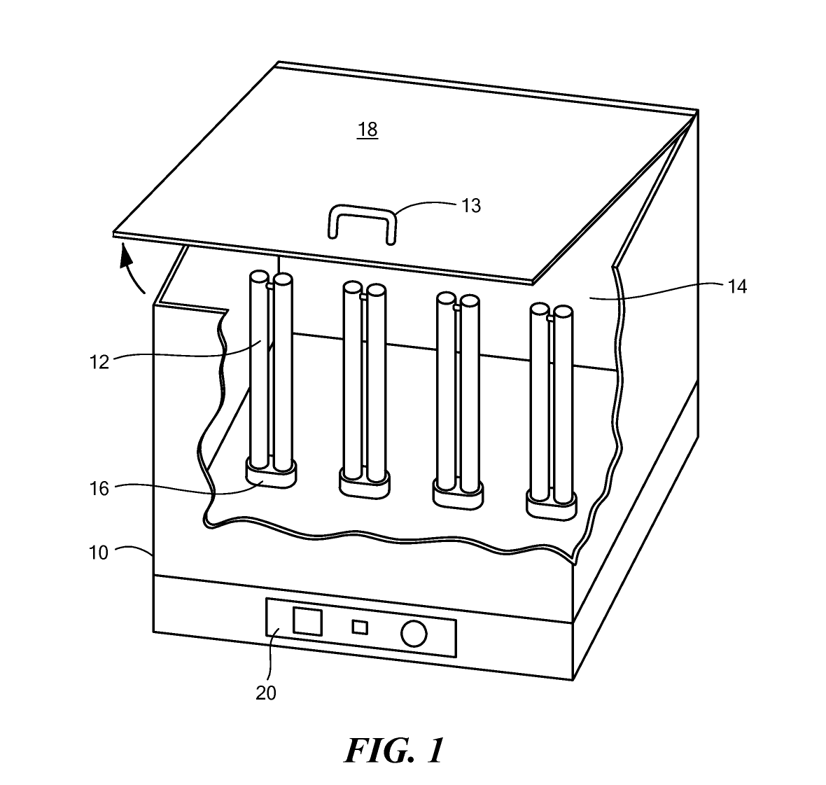

A UVC disinfection system in accordance with the invention is shown in one embodiment in FIG. 1. A housing or enclosure 10 contains a plurality of UVC lamps 12 disposed in vertical orientation in the interior chamber 14 of housing 10. In the illustrated embodiment four lamps are arranged along the width of the chamber. The lamps are in the form of U shaped tubes which are pluggable at one end into associated sockets 16 positioned in the housing 10. The lamps are typically high output, low pressure mercury or amalgam UVC generating lamps such as Light Sources Model LTC24W/2G11/FEP Coated. The lamps are typically about 12 inches in length. In the illustrated embodiment, sufficient UVC radiation is provided to kill 99% of pathogens in 30 seconds or less. The housing 10 can be made of any suitable material and in the illustrated embodiment is aluminum.

Preferably each lamp is covered by a protective sleeve to avoid shattering of the lamp quartz glass in the event of breakage. FEP (Teflon) is preferred because it is UV transmissive with little attenuation and can easily withstand the operating temperature of the lamps. The housing 10 contains a hinged top cover or door 18 and when opened such as with handle 13 permits access to the chamber 14 for placement and removal of items to be decontaminated.

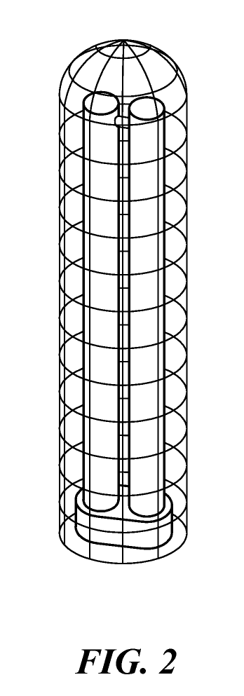

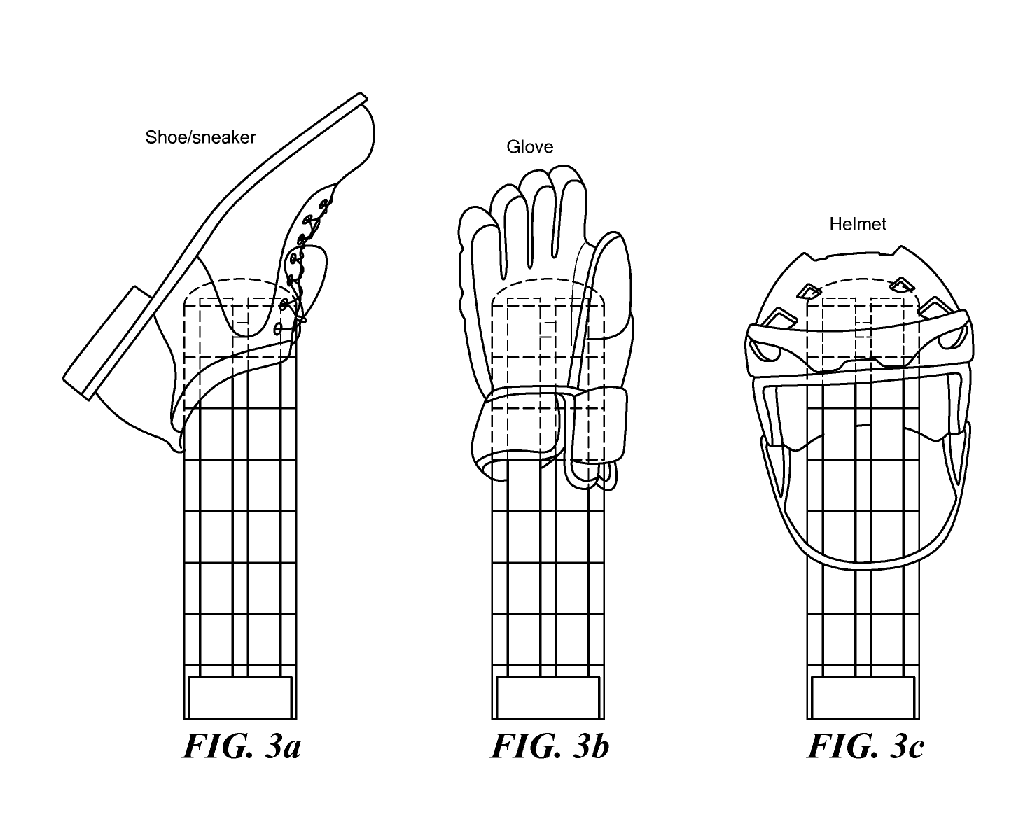

Each of the lamps 12 has an open grid, cage or basket 20 over which items to be decontaminated can be placed. The grid can be composed of metal or plastic and have a sufficiently open structure to permit emission of substantially all of the UVC radiation from the lamps. The grid is shown in FIG. 2. Each of the grids may be sized and configured to accommodate specific items such as gloves, shoes, sneakers, helmets and the like. As examples, the support grids illustrated in FIG. 3a-3c support a sneaker, glove and helmet, respectively.

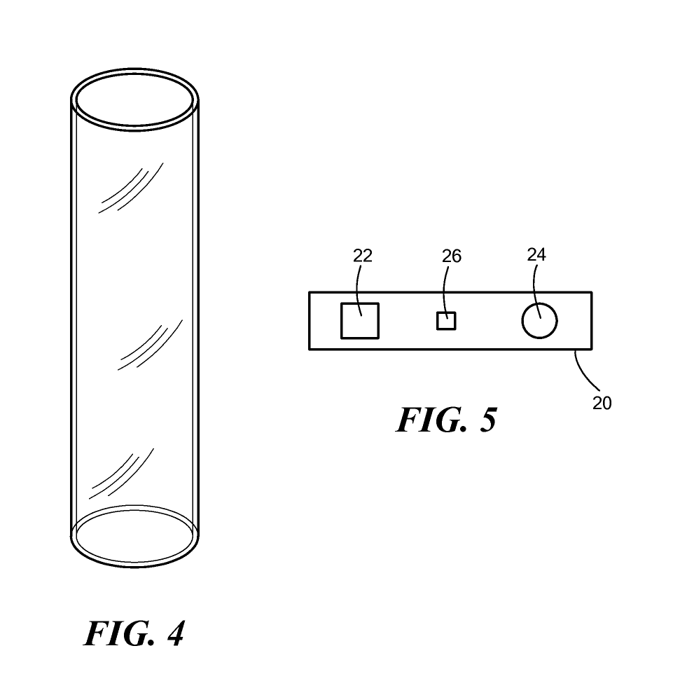

As an alternative to the grid structure, a UVC transmissive sleeve such as shown in FIG. 4, may be provided over each of the lamps to serve as a support for the items to be decontaminated. The sleeve can typically be fabricated as a hollow tube of quartz and may be of a size and shape to accommodate specific items to be decontaminated. The quartz tube can be closed at the top end and open at the bottom end in one version.

The UVC lamps are driven by a power source having electronic ballasts which start the lamps and regulate the current in each lamp to assure proper and safe operation. Each lamp may be driven by one electronic ballast or a single ballast may drive multiple lamps depending upon the particular lamps and ballasts employed. The electronic ballast may operate from a standard 110 volt 60 Hz power source or from a 220 volt 50 Hz source or from a dual voltage or other suitable source. A removable power cord may be employed for convenience of system transport.

The system is controlled by a microprocessor based microcontroller typically contained on a control board disposed within the housing. The ballasts and power components in the illustrated embodiment are disposed at the bottom of the housing behind a control panel 20. The control panel is illustrated diagrammatically in FIG. 5 and includes a display 22 such as a two digit alpha, numeric or alpha-numeric digital display to indicate countdown of remaining time during a decontamination cycle and to indicate system messages such as error conditions. An audio annunciator 24 such as a Sonalert is provided to audibly indicate, such as by a beep, that an operating cycle has ended. The annunciator can also provide distinguishable sounds to denote one or more error conditions. A start or control switch 26 is provided to activate the system. The control switch can be of the illuminated type which illuminates when actuated to start a decontamination cycle.

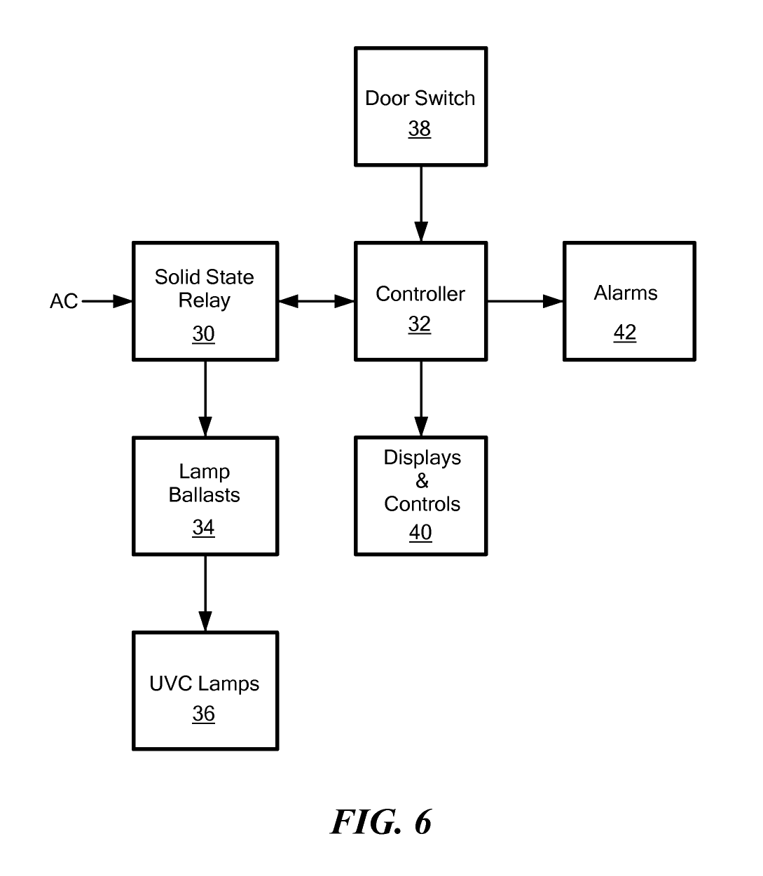

A block diagram of the system is illustrated in FIG. 6. AC input power is provided to a solid state relay 30 and thence to a controller 32 which governs system operation. The solid state relay is coupled to the lamp ballasts 34 which drive the UVC lamps 36. Over current protection devices such a fuse or circuit breaker may be provided. The controller 32 is coupled to door switch 38, to displays and controls 40 and to alarm indicator 42. The displays and controls 40 include the displays or indicators of system conditions and controls for system operation, such as those shown in the control panel of FIG. 5. The door switch 38 will cause the controller to prevent system operation or shut down operation if the door is opened during an operating cycle. The door can include a locking mechanism which prevents opening of the door during system operation.

The system typically operates for a predetermined period of time as governed by a time period set in the controller. Upon activation of the system by pushing the start switch 26, the lamps are turned on for the specified period of time and are turned off when the time period ends. The countdown of the operating time is shown in display 22 on the front panel.

The controller monitors the current to each of the electronic ballasts to insure that all of the lamps are operating properly. If the current is less than the designated reference value, the controller will turn off the UVC lamps and display a message on front panel display 22. Typically, the current monitor signal is converted to a digital signal by means of an analog to digital converter for comparison with a stored reference value. In order to determine which UVC lamp is not working properly, the system includes a diagnostic cycle by which the lamps can be turned on when the enclosure door is open. In this manner an operator can see which lamp is not working and have it replaced. A diagnostic cycle can be initiated for example by pressing the start button 26 multiple times within a designated period of time. For example, pressing the start button five times within 5 seconds will cause all of the lamps to be turned on for visual inspection.

It will be appreciated that the invention is not to be limited by the particular embodiment shown and that modifications and alternative implementations are contemplated and are within the intended scope of the invention. For example, the number and type of UVC lamps can vary and the physical configuration of the system may take different forms. Accordingly, the invention is not to be limited by what has been particularly shown and described except as defined by the appended claims.

1. An ultraviolet disinfection system for athletic items comprising:
a housing having a chamber therein and having a top, a bottom, side walls and an openable door;
one or more UVC lamps providing UVC radiation and vertically disposed in a central area of the chamber away from the walls and top;
each of the UVC lamps having a UVC transmissive enclosure surrounding the lamp and sized and configured to fit substantially inside the athletic item such that the UVC lamp is positioned substantially inside the athletic item to permit the UVC radiation to kill pathogens inside of said athletic item;
a power source having one or more ballasts for energizing the one or more UVC lamps;
an electronic controller to control operation of the system including the timing of a decontamination cycle; and
the controller also operative to monitor the current to the ballasts.

2. The system of claim 1 including a door switch associated with the door and operative to provide a signal to the controller to prevent or discontinue system operation when the door is open.

3. The system of claim 2 wherein the housing is aluminum.

4. The system of claim 1 wherein the door is hinged at the top of the housing to permit the entry and removal of items to be decontaminated.

5. The system of claim 4 wherein the door is hinged at a front wall of the housing.

6. The system of claim 1 wherein the current to the ballasts is compared to a predetermined operating level by the controller.

7. The system of claim 6 wherein the current monitor signal is converted to a digital level by means of an analog to digital converter.

8. The system of claim 7 wherein the analog to digital converter is part of a microprocessor in the controller.

9. The system of claim 1 wherein the lamps are low pressure high output mercury lamps.

10. The system of claim 1 wherein the lamps are low pressure high output amalgam lamps.

11. The system of claim 1 wherein the UVC lamps are approximately 12 inches in length.

12. The system of claim 1 wherein the UVC lamps are each U-shaped with an electrical connector on one end pluggable into a socket in the chamber.

13. The system of claim 1 wherein the power source operates at 120 VAC 60 Hertz.

14. The system of claim 1 wherein the power source operates at 220 VAC 50 Hertz.

15. (canceled)

16. The system of claim 1 wherein the controller includes a timer to set the operating time for the system.

17. The system of claim 1 where each lamp is enclosed in a protective sleeve of UVC transmissive material.

18. The system of claim 1 wherein the enclosure includes a wire grid which is sufficiently open to permit substantially all UVC radiation from the lamp to pass through.

19. The system of claim 1 wherein the enclosure includes a hollow quartz tube which permits substantially all UVC radiation from the lamp to pass through.

20. The system of claim 19 wherein one end of the quartz tube is sealed and the other end is open.

21. The system of claim 1 including a control panel on the enclosure.

22-24. (canceled)

25. The system of claim 21 wherein the control panel contains an annunciator.

26. The system of claim 25 wherein the annunciator is a Sonalert.

27. The system of claim 25 wherein the annunciator will emit one signal to indicate the end of the decontamination cycle and a different signal to indicate an error condition.

28-29. (canceled)

30. The system of claim 1 wherein the door activates a switch to indicate that the door is properly closed.

31. The switch in claim 30 wherein the switch will turn off the decontamination system if the hinged door is opened during the decontamination cycle.

32. The system of claim 1 wherein the athletic items include helmets, gloves, shoes, socks, and any other items into which the UVC transmissive enclosure can fit for decontamination by the UVC radiation.

33. The system of claim 1 wherein there is sufficient UVC intensity to kill at least 99% of Clostridium difficile in less than 30 seconds.
